(19) United States
(12) Patent Application Publication
(10) Pub. No.: US 2024/0240172 A1
(43) Pub. Date: Jul. 18, 2024

(54) Process for Purifying Nucleic Acids, in particular in a Microfluidic Apparatus

(71) Applicant: Robert Bosch GmbH, Stuttgart (DE)

(72) Inventors: Tanja Maucher, Stuttgart (DE); Christian Grumaz, Waiblingen (DE); Martina Budde, Karlsruhe (DE)

(21) Appl. No.: 18/562,025

(22) PCT Filed: Mar. 22, 2022
(86) PCT No.: PCT/EP2022/057426

§ 371 Date: Nov. 17, 2023

(30) Foreign Application Priority Data
May 17, 2021 (DE) 10 2021 204 952.4

(51) Int. Cl. C12N 15/10; B01L 3/00; C12Q 1/6806; C12Q 1/6844

(52) U.S. Cl. CPC C12N 15/1017; B01L 3/502761; C12Q 1/6806; C12Q 1/6844; B01L 2200/16; B01L 2300/0681; B01L 2300/0816

(57) ABSTRACT

A method for purifying nucleic acids, in particular in a microfluidic apparatus, is disclosed. The method includes releasing nucleic acids from a sample by way of chaotrope substances in a transport medium and binding the nucleic acids to a filter, the transport medium being mixed with a first portion of a wash buffer in order to adjust the binding conditions. A microfluidic kit for carrying out the method is further disclosed.

### PRIOR ART

In biochemical and medical diagnostics, numerous so-called transport media for viral and bacterial samples in particular have established themselves as clinical standards that make it possible to transport patient swabs or samples to a diagnostic laboratory without damaging or altering the nucleic acids in the medium. These transport media create a stabilizing environment for the nucleic acids by destroying pathogens, releasing the nucleic acids, and inhibiting degrading proteins. They are primarily used when molecular diagnostic detection of pathogens is required, but microbiological propagation on culture media such as agar plates or blood culture is not desired or possible (e.g., for virus detection).

A widely used transport medium for such a purpose is eNAT™ from the company COPAN. Another example is the Cobas® medium from the company Roche. These nucleic acid-stabilizing transport media contain so-called chaotropic salts in high concentrations (e.g., eNAT™: 3.7 M), which disrupt the structure of water, but also of macromolecules dissolved in water, such as proteins or nucleic acids, and can lead to denaturation of the structures. Lipid bilayers, such as those found in cellular membranes, can also be denatured. This effect leads to the lysis of a wide variety of cells, such as human cells, bacteria, and also to the disruption of the protein envelope of viruses.

The classic sample purification method commonly used in a lab-on-chip system, comprising cell lysis, binding of the released nucleic acids to a filter frit, washing and subsequent elution of the purified nucleic acids with subsequent (real-time) polymerase chain reaction (PCR for short) and detection reaction, as described for example in the disclosure DE 10 2014 211 221 A1, can in principle be carried out with these transport media. In a first step, a so-called lysis or binding buffer is used for cell lysis and binding of the nucleic acids to the filter frit. This buffer is formulated in such a way that the concentration of chaotropic salts in the mixed compound is optimally adjusted for binding the nucleic acids to the filter frit. This optimal range is approximately 1.6-2.2 moles per liter (Molar or M for short). In addition, this binding buffer usually contains reagents that promote the precipitation of nucleic acids. These are often other salts (e.g., common salt) or alcohols (e.g., isopropanol). The adjustment of these chaotropic conditions is essential for the recovery rate of nucleic acids.

In a second step, cell and protein residues as well as salts and precipitating agents are removed as completely as possible by using a wash buffer. This is necessary to prevent inhibition of the detection reaction by (quantitative real-time) PCR by these substances. The formulation of the wash buffer must again be carefully selected in order to sufficiently remove interfering substances without rinsing the nucleic acids from the filter and thus losing them for the detection reaction. A cascade of differently concentrated solutions with chaotropic salts and precipitating agents is often used in succession instead of a single wash buffer.

In a third step, the nucleic acids are dissolved from the filter frit with a so-called elution buffer and added to the detection reaction. For example, a freeze-dried PCR reaction mix is rehydrated with the elution buffer and the nucleic acids it contains, which can then be cycled in a classic PCR temperature program.

### DISCLOSURE OF THE INVENTION

### Advantages of the Invention

Against this background, the invention relates to a method for purifying nucleic acids. The method can be carried out in particular with a microfluidic apparatus, preferably a so-called lab-on-a-chip, for example with a microfluidic cartridge as described in DE 10 2016 222 075 A1 or DE 10 2016 222 072 A1.

In a first step of the method, nucleic acids are released from a sample in a transport medium due to chaotropic substances in the transport medium. The transport medium can thus be a medium for storing, preserving, and/or transporting biological samples, in particular for samples of body fluids containing human or animal cells. The transport medium contains chaotropic substances, in particular chaotropic salts, for releasing nucleic acids from the cells. For example, the above-mentioned transport media are eNAT™ or Cobas®. The release can take place in particular by lysis of the cells in the sample, wherein the cells comprise the nucleic acids. Furthermore, the release may comprise denaturation of the nucleic acids. Nucleic acids can be understood in particular as ribonucleic acids (RNA for short) or deoxyribonucleic acids (DNA for short). The chaotropic substances may in particular be chaotropic salts, such as barium salts, guanidinium hydrochloride, thiocyanates such as guanidinium thiocyanate, perchlorates such as sodium perchlorate or sodium chloride.

In a second step of the method, nucleic acids are bound to a filter, wherein the transport medium is mixed with a first portion of a wash buffer to adjust the binding conditions. The mixing creates a binding buffer to bind the nucleic acids to the filter. The filter can be a filter of the microfluidic apparatus in particular. The filter can be based on a porous silica membrane, for example. Furthermore, a filter can preferably also be understood as a filter frit. The wash buffer may be a buffer or solution commonly used for the purification of nucleic acids, but preferably without ethanol and preferably containing less than 0.1 mole per liter of chaotropic substances, in particular chaotropic salts. Preferably, by mixing the transport medium with the first portion of the wash buffer, a concentration of the chaotropic substances for adjusting the binding conditions is lowered, preferably creating a binding buffer with a concentration of chaotropic substances between 1.6 to 2.2 moles per liter. When using chaotropic salts as chaotropic substances, mixing the transport medium with the first portion of the wash buffer preferably creates a mixture containing chaotropic salts with a concentration of between 1.6 and 2.2 moles per liter. Furthermore, according to an advantageous embodiment, the first portion of the wash buffer has highly crosslinked polyalcohols, such as polyethylene glycols, so that the mixture preferably has these polyalcohols in a proportion of 150 to 200 grams per liter (g/L for short). Preferably, the wash buffer has a proportion of 200 to 400 g/L of highly crosslinked polyalcohols. In other words, the first portion of the wash buffer has such an amount of chaotropic substances, preferably chaotropic salts, and preferably highly crosslinked polyalcohols, that mixing the first portion of the wash buffer with a predetermined amount of the predetermined transport medium creates a mixture which contains 1.6 to 2.2 moles per liter of chaotropic substances, the chaotropic substances preferably being chaotropic salts, and 150 to 200 grams per liter of highly crosslinked polyalcohols, the highly crosslinked polyalcohols preferably being polyethylene glycol.

The method according to the invention has the advantage that an otherwise necessary binding buffer for binding the nucleic acids to the filter can be dispensed with, since this binding buffer is replaced by mixing the transport medium with the first portion of the wash buffer. In other words, by mixing the transport medium with the first portion of the wash buffer according to the invention, a binding buffer is created for binding the nucleic acids to the filter when carrying out the method. The resulting elimination of a separate binding buffer advantageously leads to cost savings both in the procurement and formulation of reagents and buffers and in the design and manufacture of a microfluidic apparatus used for this purpose, as space for a receptacle and pre-storage, and thus material, can be saved.

According to a preferred further development of the invention, the purification of the nucleic acids after binding to the filter comprises washing the filter with a second portion of the wash buffer. This has the advantage that substances remaining on the filter or in the filter chamber, for example cell and protein residues and, in particular, salts and precipitating agents, are removed and therefore cannot adversely affect the rest of the method. In particular, substances that would negatively affect the polymerase chain reaction can be removed. The first portion and the second portion of the wash buffer can preferably be two portions of the same wash buffer.

According to a preferred further development of the method, the nucleic acids are eluted from the filter using an elution buffer. The elution buffer can be a buffer typically used in microfluidics for the elution of nucleic acids, for example buffered low-salt solutions with a neutral to slightly alkaline pH value (for example TE buffer 10 mM of TRIS/Cl pH 8, 1 mM of EDTA) or distilled water.

In a particularly advantageous further development of the method, portions of the nucleic acids are amplified after binding, in particular after elution, preferably using a (quantitative real-time) polymerase chain reaction, an isothermal amplification, or a ligase chain reaction. A portion of the nucleic acids can be understood as a section, also known as a sequence, of a nucleic acid, for example a DNA or RNA section. Subsequently or in parallel to the multiplication, these portions, and thus the associated organisms, can be detected by detecting the multiplied parts, for example with the aid of (fluorescence) spectroscopy.

An object of the invention is also a microfluidic kit or microfluidic system comprising a microfluidic apparatus and a transport medium, wherein the transport medium contains chaotropic substances for releasing nucleic acids from a sample and wherein the apparatus is configured to mix a first portion of a wash buffer with the transport medium for adjusting the binding conditions for binding the released nucleic acids to a filter of the apparatus. The wash buffer can be located upstream in the microfluidic apparatus. The transport medium can be a medium as explained above, in particular eNAT™ or Cobas®. In particular, the microfluidic kit can be understood to mean the microfluidic apparatus together with the transport medium, wherein the transport medium does not have to be in the microfluidic apparatus. The transport medium can, for example, be arranged in a vessel, wherein the sample can also be added to the vessel for mixing with the transport medium, for example by inserting a swab containing the sample into the transport medium.

Preferably, the transport medium and the first portion of the wash buffer are matched with respect to their respective amount, their composition, and a concentration of the chaotropic substances in such a way that the transport medium is adapted to release nucleic acids from a sample and that mixing of the transport medium with the first portion of the wash buffer is configured to adjust the binding conditions for binding the released nucleic acids to the filter. As described above, binding conditions are present in particular if the mixture contains a concentration of between 1.6 and 2.2 moles per liter of chaotropic substances, preferably chaotropic salts, and most preferably 150 to 200 grams per liter of highly crosslinked polyalcohols, the highly crosslinked polyalcohols preferably being polyethylene glycol.

According to a particularly preferred embodiment, the kit, in particular the microfluidic apparatus, comprises a second portion of the wash buffer, wherein the second portion is designed to wash the filter and the nucleic acids bound to the filter. Preferably, the second portion has the same composition as the first portion of the wash buffer.

For further embodiments and advantages of the microfluidic kit or system, reference is made to the above comments on the method.

### BRIEF DESCRIPTION OF THE DRAWINGS

Exemplary embodiments of the invention are shown schematically in the drawings and explained in more detail in the following description. The same reference signs are used for the elements having the same effect and shown in the various drawings, so repeated description of these elements is omitted.

### Shown are:

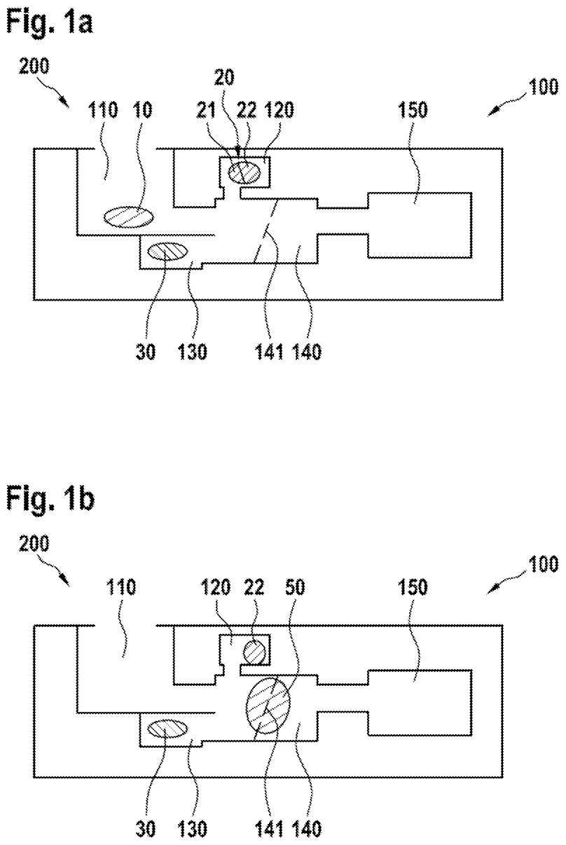

FIGS. 1a, 1b exemplary embodiments of the kit according to the invention and

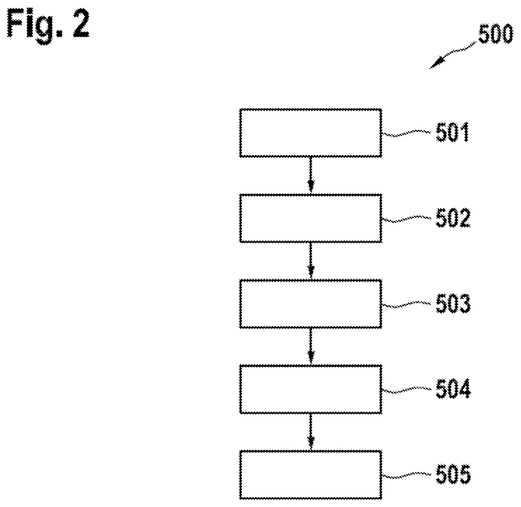

FIG. 2 a flow diagram of an exemplary embodiment of the method according to the invention.

### EMBODIMENTS OF THE INVENTION

FIGS. 1a and 1b show an exemplary embodiment of the microfluidic kit 200 according to the invention, with which the method 500 according to the invention can be carried out, for example. FIG. 2 shows a flow diagram 500 of the following described exemplary embodiment of the method 500 according to the invention.

The microfluidic kit 200 comprises a microfluidic apparatus 100, for example a microfluidic cartridge 100 as a portion of a lab-on-a-chip apparatus, as described in DE 10 2016 222 075 A1 or DE 10 2016 222 072 A1.

A biological sample is introduced in a transport medium 10 via an opening 111 into an input chamber 110 of the cartridge 100. The biological sample may in particular be a body fluid such as blood, sputum, urine, or a swab containing human or animal cells.

The transport medium 10 comprises chaotropic substances for releasing the nucleic acids from the cells according to a step 501 of the method 500. In particular, the transport medium 10 can be eNAT™ from the company COPAN or Cobas® from the company Roche. The transport medium 10 contains, for example, approx. 3.7 moles per liter (molar or M for short) of guanidinium thiocyanate as a chaotropic salt and a total volume of, for example, 300 microliters (μL for short).

As shown in FIG. 1a, the cartridge 100 comprises a wash buffer 20, which can be stored in a chamber 120 (wash buffer chamber 120 for short) upstream of the cartridge 100. The wash buffer 20 is preferably a buffer without chaotropic substances or with only a low concentration of chaotropic substances, preferably with a concentration of less than 0.1 M. For example, it is a wash buffer without ethanol, as described in EP 2 163 621 A1, and for example comprising polyethylene glycol in a concentration between 100 and 600 g/L as described in DE 10 2014 211 221 A1.

The cartridge 100 further comprises a filter 141 fluidically connected to the input chamber 110 and the buffer chamber 120, which can be arranged in a further chamber 140 (filter chamber 140 for short). According to a second step 502 of the method 500, the released nucleic acids are bound to the filter 141, for which purpose the transport medium 10 is mixed with a first portion 21 of the wash buffer 20 to adjust suitable binding conditions and the filter 141 is exposed to this mixture, which now corresponds to a binding buffer 50, for a period of time of, for example, 15 to 60 seconds, as shown in FIG. 1b. The first portion 21 of the wash buffer 10 provided for use in combination with the transport medium 10 is designed or formulated in such a way that, in addition to adjusting the binding conditions after mixing with the transport medium 10, it can also be used in its function as a wash buffer for the subsequent removal of chaotropic substances and other substances inhibiting subsequent analysis, in particular for carrying out a (quantitative real-time) polymerase chain reaction. In particular, as explained above, the wash buffer is designed such that it contains few or no chaotropic substances and at the same time, highly crosslinked polyalcohols such as polyethylene glycols to support nucleic acid precipitation (for example in the range of 20 to 40% (w/v)). Thus, the wash buffer 20 can advantageously be used, both after mixing with the transport medium 10 as a binding buffer for binding and as an ordinary wash buffer for displacing the chaotropic substances in a washing step.

eNAT™ contains about 3.7 M guanidinium thiocyanate as a chaotropic salt as a main component that determines the binding properties for binding the nucleic acids to the filter 141. For example, 300 microliters of eNAT™ are also used for the detection of various pathogens. The final concentration of chaotropic salts for binding the nucleic acids to the filter 141 should be between 1.6 and 2.2 M. By diluting the eNAT™ with the first portion 21 of the wash buffer 20, these suitable binding conditions are achieved if the wash buffer contains no or few chaotropic substances as described above.

In a preferably third step 503 of the method 500, the filter 141 and the nucleic acids bound thereto are washed with a second portion 22 of the wash buffer 20 to remove residues of the binding buffer 50 and other substances from the sample and the transport medium 10, in particular remaining chaotropic substances, which would have a detrimental effect on the further method.

In a fourth step 504 of the method 500, an elution buffer 40 is used to remove the binding conditions and thus detach the nucleic acids from the filter 141. For this purpose, the elution buffer 30 can be stored upstream in a further chamber 130 (elution buffer chamber 130 for short) fluidically connected to the filter chamber 140. The elution buffer can be a typical buffer for dissolving nucleic acids from silica, for example distilled or buffered water, possibly with surface-active substances such as polysorbate 80 (Tween® 80).

The purification of the nucleic acids is completed with the fourth step 504 and further processing of the nucleic acids can take place in a preferably fifth step 505, for example amplification of portions of the nucleic acids with the aid of a (quantitative real-time) polymerase chain reaction, an isothermal amplification, or a ligase chain reaction in a further chamber 150 (analysis chamber 150 for short) for detection of the nucleic acids and thus associated pathogens in the sample.

1. A method for purifying nucleic acids in a microfluidic apparatus, comprising:
releasing nucleic acids from a sample by chaotropic substances in a transport medium; and
binding the nucleic acids to a filter, wherein the transport medium is mixed with a first portion of a wash buffer to adjust the binding conditions.

2. The method according to claim 1, further comprising washing the filter after binding the nucleic acids with a second portion of the wash buffer.

3. The method according to claim 1, wherein after binding an elution of the nucleic acids from the filter is carried out using an elution buffer.

4. The method according to claim 1, wherein by mixing the transport medium with the first portion of the wash buffer, a concentration of the chaotropic substances for adjusting the binding conditions is lowered.

5. The method according to claim 1, wherein mixing the transport medium with the first portion of the wash buffer creates a mixture having a concentration of chaotropic substances.

6. The method according to claim 1, wherein by mixing the transport medium with the first portion of the wash buffer, a mixture is created which comprises highly crosslinked polyalcohols.

7. The method according to claim 1, wherein after the binding, a polymerase chain reaction, an isothermal amplification, or a ligase chain reaction for amplifying at least a portion of the nucleic acids takes place.

8. A microfluidic kit comprising a microfluidic apparatus and a transport medium, wherein the transport medium contains chaotropic substances for releasing nucleic acids from a sample, and wherein the apparatus is configured to mix a first portion of a wash buffer with the transport medium for adjusting the binding conditions for binding the released nucleic acids to a filter of the apparatus.

9. The kit according to claim 8, wherein the transport medium and the first portion of the wash buffer are matched with respect to their respective amount, their composition, and a concentration of the chaotropic substances such that the transport medium is adapted to release nucleic acids from a sample and that mixing of the transport medium with the first portion of the wash buffer is configured to adjust the binding conditions for binding the released nucleic acids to the filter.

10. The kit according to claim 8, wherein a second portion of the wash buffer has the same composition as the first portion of the wash buffer, and wherein the second portion is designed for washing the filter and the nucleic acids bound to the filter.

11. The method according to claim 5, wherein:
the chaotropic substances include chaotropic salts, and
the concentration of chaotropic substances is between 1.6 to 2.2 moles per liter.

12. The method according to claim 6, wherein:
the highly crosslinked polyalcohols include polyethylene glycols, and
the mixture is created which comprises highly crosslinked polyalcohols in a proportion of 150 to 200 grams per liter of the mixture.

13. The method according to claim 1, wherein after the elution, a polymerase chain reaction, an isothermal amplification, or a ligase chain reaction for amplifying at least a portion of the nucleic acids takes place.
